(19) United States
(12) Patent Application Publication
(10) Pub. No.: US 2016/0158562 A1
(43) Pub. Date: Jun. 9, 2016

(54) SYSTEMS AND METHODS FOR NEUROSTIMULATION OF A PERIPHERAL NERVE

(71) Applicant: PACESETTER, INC., Sylmar, CA (US)

(72) Inventors: Gene A. Bornzin, Simi Valley, CA (US); Timothy A. Fayram, Gilroy, CA (US); Stuart Rosenberg, Castaic, CA (US); Zoltan Somogyi, Simi Valley, CA (US); John W. Poore, South Pasadena, CA (US)

(21) Appl. No.: 14/927,328

(22) Filed: Oct. 29, 2015

Related U.S. Application Data
(60) Provisional application No. 62/089,705, filed on Dec. 9, 2014.

(51) Int. Cl. A61N 1/372; A61N 1/36; A61N 1/05

(52) U.S. Cl. CPC A61N 1/37223; A61N 1/0551; A61N 1/36017; A61N 1/36021

(57) ABSTRACT

Systems and methods are provided for neurostimulation (NS) of peripheral nerves and/or associated ganglion. The systems and methods create a magnetic field from an elongated transmission coil of an external stimulator and expose an elongated receiver coil of a magnetic driver to the magnetic field. The systems and methods generate at the magnetic driver a pulse forming a stimulation waveform in response to the magnetic field. The systems and methods deliver the stimulation waveform to a target peripheral nerve through an electrode from the magnetic driver.

### CROSS REFERENCE TO RELATED APPLICATIONS

This application relates to and claims priority benefits from U.S. Provisional Application No. 62/089,705, filed Dec. 9, 2014, entitled “Magnetically Coupled Stimulator Using Elongated Wire Coils,” which is hereby incorporated by reference in its entirety.

### BACKGROUND OF THE INVENTION

A percentage of individuals that suffer from intractable chronic headaches, such as chronic migraine and chronic cluster headaches, have repeating symptoms every few days or more each month. Neurostimulation (NS) systems have recently been used for treatment of chronic migraine and chronic cluster headaches by stimulating peripheral nerves.

NS systems are devices that generate electrical pulses, and deliver the pulses to nerve tissue to treat a variety of disorders via one or more electrodes. While a precise understanding of the interaction between the applied electrical energy and the nervous tissue is not fully appreciated, it is known that application of electrical pulses depolarize neurons and generate propagating action potentials into certain regions or areas of nerve tissue. The propagating action potentials effectively mask certain types of physiological neural activity, increase the production of neurotransmitters, or the like.

Conventional NS systems stimulate peripheral nerves such as the sphenopalatine ganglion (SPG) under the maxillary bone or via the gums of the lower jaw to treat cluster headaches. These conventional NS systems include a large stimulator structure, such as a disc or coin shape, implanted within the patient. The large stimulator structure includes dedicated ASICs for stimulating the peripheral nerve targets. However, due to the size of the large stimulator structure, the large stimulator is not injectable into the patient and instead requires a large pocket for implantation. Accordingly, new systems and methods are needed for a simple, low profile, subcutaneous stimulator of an NS system to stimulate peripheral nerves and/or associated ganglion.

### SUMMARY

In accordance with one embodiment, a neurostimulation (NS) system is described with an external stimulator having an elongated transmission coil configured to generate a magnetic field. The external stimulator controls a field characteristic of the magnetic field in connection with a stimulation waveform. The NS system further includes a lead implantable within a patient. The lead having a magnetic driver and an electrode. The magnetic driver being electrically coupled to the electrode. The magnetic driver including an elongated receiving coil that extends along an axis of the lead. When the magnetic driver is exposed to the magnetic field, the magnetic driver generates a pulse forming the stimulation waveform to be delivered through the electrode to a target peripheral nerve.

In an embodiment, a method for neurostimulation of peripheral nerve fibers is described. The method may include creating a magnetic field from an elongated transmission coil of an external stimulator. The external stimulator controls a field characteristic of the magnetic field in connection with a stimulation waveform. The method may further include exposing an elongated receiver coil of a magnetic driver to the magnetic field, and generating at the magnetic driver a pulse forming the stimulation waveform in response to the magnetic field. The method may also include delivering the stimulation waveform to a target peripheral nerve through an electrode from the magnetic driver, which is electrically coupled to the electrode.

### BRIEF DESCRIPTION OF THE DRAWINGS

FIG. 1 illustrates a schematic diagram of a lead for stimulating peripheral nerves and/or associated ganglion, in accordance with an embodiment of the present disclosure.

FIG. 2 illustrates an exploded view of the lead shown in FIG. 1.

FIG. 3 illustrates a lead for stimulating a peripheral nerve, in accordance with an embodiment of the present disclosure.

FIG. 4 illustrates a schematic diagram of an external stimulator, in accordance with an embodiment of the present disclosure.

FIG. 5 illustrates a flow chart of a method for neurostimulation of a peripheral nerve fiber, in accordance with an embodiment of the present disclosure.

FIG. 6 illustrates a position of a lead with respect to patient, in accordance with an embodiment of the present disclosure.

FIG. 7 is a graphical representation of a current signal received by an elongated transmission coil and a stimulation waveform received from an elongated receiver coil resulting from the current signal, in accordance with an embodiment.

FIG. 8 illustrates a functional block diagram of a portable device, in accordance with an embodiment of the present disclosure.

FIG. 9 illustrates an electrical circuit diagram of an external stimulator receiving attributes of a stimulation waveform, in accordance with an embodiment of the present disclosure.

FIG. 10 illustrates a schematic diagram of a neurostimulation system, in accordance with an embodiment of the present disclosure.

### DETAILED DESCRIPTION

While multiple embodiments are described, still other embodiments of the described subject matter will become apparent to those skilled in the art from the following detailed description and drawings, which show and describe illustrative embodiments of disclosed inventive subject matter. As will be realized, the inventive subject matter is capable of modifications in various aspects, all without departing from the spirit and scope of the described subject matter. Accordingly, the drawings and detailed description are to be regarded as illustrative in nature and not restrictive.

Various embodiments described herein include methods and/or systems for stimulation of peripheral nerves which may include associated ganglion from a lead. The lead may be a low profile subcutaneous stimulator. The lead may be used for any peripheral nerve stimulation application, for example, treatment of headaches. The stimulator may have a small diameter coil (e.g., two to three millimeters) over a ferrite rod (e.g., diameter of one to two millimeters), and one or more small diameter tubes (e.g., one half or one millimeter diameter) extending from the ferrite rod. The size of the components (e.g., the coil, the ferrite rod, tubes) allow the lead to have a low profile less than ten millimeters, such as three and a half millimeters. For example, the low profile of the lead enables the lead to be positioned between the skin and the skull of the patient without being readily apparent.

The lead may include one or more unipolar, bipolar, or multipolar electrodes. Optionally, one or more of the electrodes may be pulled to an implant site using a subcutaneous suture. The stimulator may be used for episodic pain like headaches, migraines, and/or for a time limited pain or for cancer pain that resolves with therapy or because the patient is terminal. Time limited pain, for example, may include fathom limb pain that ultimately tends to resolve with time.

The lead may be implanted using a rapid, minimal subcutaneous procedure. The clinician (e.g., doctor, implanter) may localize an implant site by using an insulated needle connected to an external trial stimulator to localize the target ganglion. Implantation may be performed in an alert patient without anesthesia. The verification of the implant site may be achieved based on patient feedback.

During implantation, for example, a suture (e.g., a No. 2 suture, a No. 3 suture) may be tunneled under the skin of the patient to a location behind the ear. The electrode may be tied to the suture then the lead may be pulled under the skin until the electrode reaches the implant site. The lead may then be inductively coupled to the implant via the electrodes. The target location may be verified by stimulating the proximate nerves via emitting one or pulses forming a stimulation waveform. Once the implant site is verified, the coil may be implanted through an incision (e.g., three millimeter long incision) and closed with tissue adhesive.

A technical effect of the low profile subcutaneous stimulator or lead is a smaller size relative to conventional NS systems. Additionally, due to the smaller size of the low profile subcutaneous stimulator, formation of a pocket for the low profile subcutaneous stimulator is not required during implantation. A technical effect of the low profile subcutaneous stimulator is reduced manufacturing cost relative to conventional NS systems. A technical effect of the low profile subcutaneous stimulator is increasing the ease in removing a subcutaneous stimulator due to the small size and the superficial, subcutaneous location of the coil.

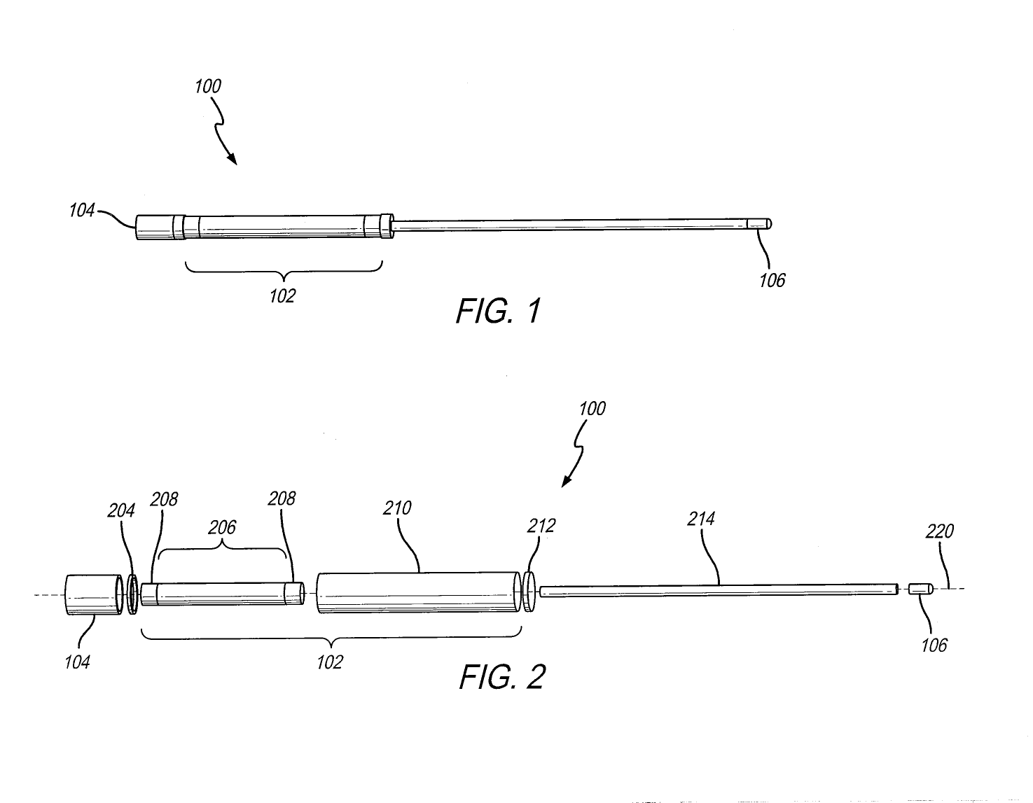

FIGS. 1 and 2 illustrate a lead 100 such as a low profile hermetically sealed subcutaneous stimulator for stimulating a target peripheral nerve. FIG. 2 illustrates an exploded view of the lead 100 shown in FIG. 1. The target peripheral nerve may correspond to nerves and/or ganglia outside of the brain and/or spinal cord. For example, the target peripheral nerves may include the occipital nerve, the supraorbital nerve, the trigeminal nerve, and/or the cervical nerve. Optionally, the peripheral nerves may be associated with a peripheral nerve ganglion such as the sphenopalatine ganglion. Additionally or alternatively, the target peripheral nerve may be positioned below the neck, for example, at a peripheral nerve within a forearm, leg, back, and/or the like. Additionally or alternatively, in various embodiments the lead 100 may be configured (e.g., by adjusting a length of the lead 100) to stimulate a less peripheral nerve, such as the vagus, a nerve within the brain, within the epidural space on the surface of the brain, on the dorsal root ganglion, and/or the like.

The lead 100 includes a magnetic driver 102 and one or more electrodes 104 and 106. It should be noted in other embodiments, the lead 100 may include more than one magnetic driver 102 each electrically coupled to different electrodes. The magnetic driver 102 may be configured to generate an electric current and voltage in response to being exposed to a varying magnetic field. The electric current may be passed through an elongated receiving coil (ERC) 206.

The ERC 206 may be an electrical conductor or a wire (e.g., thirty gauge to forty five gauge) composed of an electrically conductive material such as copper, gold, graphene, aluminum, nickel, and/or the like. The ERC 206 may have, for example, a diameter of about one to three millimeters and a length of about three to twenty millimeters in length. The ERC 206 may be a coil or winding that extends along an axis 220 of the lead 100. The ERC 206 may include multiple loops or turns (e.g., four hundred to two thousand turns) positioned at different points along the axis 220 forming, for example, a solenoid. The turns allow the ERC 206 to extend along the axis 220. Optionally, the ERC 206 may be helically wound about a rod 208. The turns may be electrically isolated from each other. For example, a void or space may be interposed between successive turns to prevent current from passing between the turns. In another example, an insulator such as a plastic or enamel may be positioned between the successive loops to electrically isolate the turns.

The rod 208 may be configured to increase a magnitude of a magnetic field formed around the ERC 206 by providing a core for the ERC 206. For example, the ERC 206 may be wound about the outer surface area of the rod 208. The rod 208 may be composed of a ferrous material such as iron compounds or alloys, ferrites, and/or the like. The rod 208 may have a cylindrical shape with a diameter of one to two millimeters. It should be noted that in other embodiments the rod 208 may be larger than two millimeters (e.g., two and half millimeters, three millimeters).

The magnetic driver 102 may include a rear ring 204 and a front cover 212 coupled to opposing ends of a tube 210 that form an enclosure around the ERC 206 and the rod 208. For example, the rear ring 204, the front cover 212 and the tube 210 may be configured to hermetically seal the magnetic driver 102. The rear ring 212 and front cover 212 may be coupled to the tube 210 by brazing the rear ring 212 and the front cover 212 to the tube 210. The rear ring 204 may be composed of Niobium and coupled to the electrode 104. The front cover 212 may be composed of Niobium and coupled to an elongated tube 214. The tube 210 may be composed of sapphire extending along the axis 220 having a length approximately the same as the rod 208.

Additionally or alternatively, the magnetic driver 102 may be enclosed using a potting process. For example, the magnetic driver 102 may be enclosed using silicone, epoxy, and/or the like to protect the ERC 206 from the tissue and/or body fluids of the patient when implanted.

The elongated tube 214 may have a cylindrical shape, for example, with a diameter of about a half millimeter. The elongated tube 214 may be composed of an insulative material and/or biocompoatible material to allow the elongated tube 214 to be implantable within the patient. Non-limiting examples of such materials include polyurethane, polyimide, polyetheretherketone (PEEK), polyethylene terephthalate (PET) film (also known as polyester or Mylar), polytetrafluoroethylene (PTFE) (e.g., Teflon), or parylene coating, and/or polyether bloc amides. The elongated tube 214 may be coupled to the front cover 212 and the electrode 106 at opposing ends of the elongated tube 214. The elongated tube 214 may house an electrical conductor (e.g., a wire) extending from the magnetic driver 102 to the electrode 106.

Although not required for all embodiments, the elongated tube 214 may be fabricated to flex and elongate upon implantation or advancing within the tissue of the patient towards the target peripheral nerve and movements of the patient during and/or after implantation. Optionally, the elongated tube 214 or a portion thereof is capable of elastic elongation under relatively low stretching forces. Also, after removal of the stretching force, the elongated tube 214 may be capable of resuming its original length and profile. For example, the elongated tube 214 may stretch 10%, 20%, 25%, 35%, or even up or above to 50% at forces of about 0.5, 1.0, and/or 2.0 pounds of stretching force.

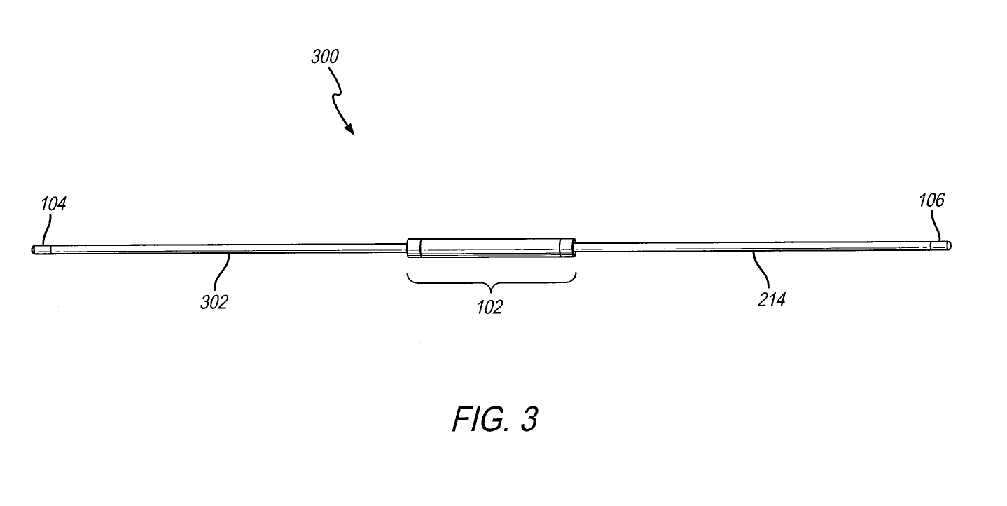

Optionally, in connection with FIG. 3, a lead 300 may have multiple elongated tubes 214, 302. FIG. 3 illustrates an alternative lead 300 having two elongated tubes 214 and 302. The elongated tube 302 may be similar to and/or the same as the elongated tube 214. The elongated tube 302 may be coupled to the electrode 104 and the rear ring 204 or a rear cover (not shown) at opposing ends of the elongated tube 302. The rear cover may be similar to and/or the same as the front cover 212. The elongated tube 302 may house an electrical conductor (e.g., a wire) extending from the magnetic driver 102 to the electrode 104.

The elongated tubes 214 and 302 may increase the affective stimulation area of the lead 300 relative to the lead 100, by allowing the pulses emitted from the lead 300 to stimulate target peripheral nerves positioned at opposing locations a greater distance from the magnetic driver 102. For example, the elongated tube 214 may be positioned and/or oriented such that the electrode 106 is positioned proximate to the occipital nerve, and the elongated tube 302 may be positioned and/or oriented such that the electrode 104 is positioned proximate to the supraorbital nerve.

Returning to FIG. 2, electrical connectors may couple the ERC 206 to the electrodes 104 and 106, allowing the magnetic driver 102 to be electrically coupled to the electrodes 104 and 106. For example, the rear ring 204 and the elongated tube 214 may include an insulative material about one or more conductors within the material that extends from the ERC 206 to the electrodes 104 and 106, respectively. Thereby, one or more pulses from the ERC 206 are provided to the electrodes 104 and 106. The pulses forming the stimulation waveform may then be applied to the target peripheral nerve of a patient via the electrodes 104 and 106. The stimulation waveform may be configured, having pre-determined attributes (e.g., amplitude, frequency), to relieve symptoms of the patient by stimulating the target peripheral nerve. For example, the stimulation waveform may be configured to relieve a migraine or headache.

The electrodes 104 and 106 may be positioned along the axis 102 of the lead 100. The electrodes 104 and 106 may be composed of an electrically conductive alloy such as titanium, platinum, and/or the like. The electrodes 104 and 106 may be in the shape of a lid such that each electrode 104 and 106 continuously covers the circumference and ends of the exterior surface of the lead 100. For example, the electrodes 104 and 106 may have a diameter of a half millimeter. Additionally or alternatively, the electrodes 104 and 106 may be in the shape of a ring. The electrodes 104 and 106 may be configured to emit the pulses in an outward radial direction proximate to or within a stimulation target. Additionally or alternatively, the electrodes 104 and 106 may be in the shape of a split or non-continuous ring such that the pulse may be directed in an outward non-uniform radial direction adjacent to the electrodes 104 and 106. It should be noted that although the lead 100 is depicted with two electrodes 104 and 106, the lead 100 may include any suitable number of electrodes 104 and 106 (e.g., more than two). Optionally, the electrode 104 may be the same and/or different size than the electrode 106. For example, the electrode 104 may have a larger diameter than the electrode 106.

Additionally or alternatively, the electrodes 104 and 106 may be configured in a cathode state (e.g., electrically coupled to the common ground of the magnetic driver 102) or an anode state such that current is emitted from the electrode in the anode state to the electrode in the cathode state. For example, in connection with the lead 100, the electrode 104 may be configured in an anode state and the electrode 106 may be configured in a cathode state.

A magnetic field may provide energy to the magnetic driver 102 to generate the one or more pulses forming the stimulation waveform. For example, when the ERC 206 or generally the magnetic driver 102 is exposed to a magnetic field, current and/or voltage is induced within the ERC 206. The characteristics of the pulses may be defined by at least one pulse characteristic that is based on characteristics of the magnetic field. For example, variances in strength and/or direction of the magnetic field over time may define an amplitude, pulse width, number of pulses, and/or frequency of pulses generated by the magnetic driver 102.

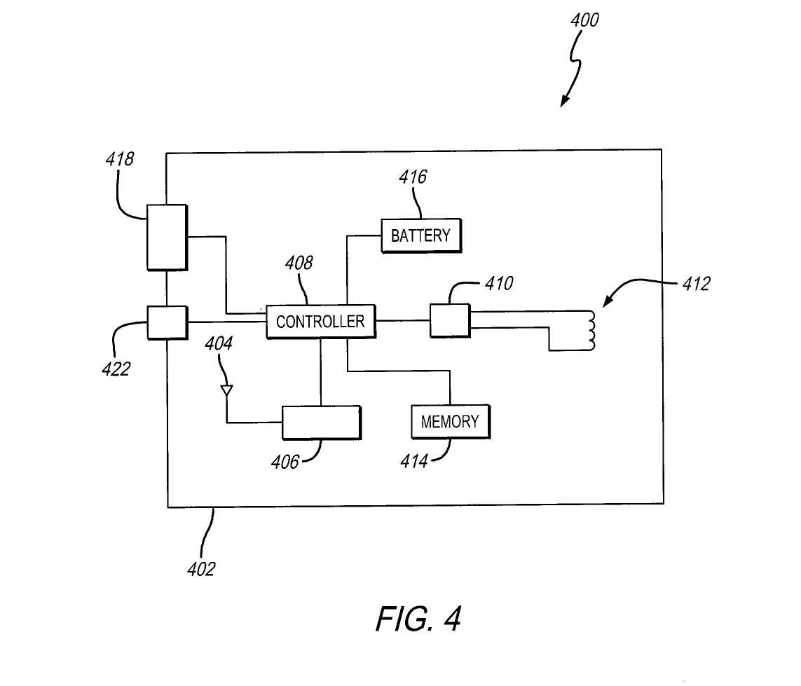

In connection with FIG. 4, the magnetic driver 102 may be exposed to a magnetic field generated by an external stimulator 400, which magnetically and/or inductively couples the lead 100, 300 to the external stimulator 400.

FIG. 4 illustrates a schematic diagram of the external stimulator 400 that generates a magnetic field. The external stimulator 400 typically includes a housing 402 that encloses a controller 408, an elongated transmission coil (ETC) 412, a power source (e.g., a battery) 416, an RF circuit 406, an antenna 404, generating circuitry 410, memory 414 (e.g., a tangible and non-transitory computer readable storage medium, such as ROM, RAM, EEPROM, and/or the like). The power source 416 provides operating power to the controller 408 and other components of the external stimulator 400. Optionally, the antenna 404 and/or the ETC 412 may be positioned on the exterior surface of the housing 402.

The housing 402 may be composed of a plastic and/or other non-conductive material. The housing 402 may be configured to be handheld by the patient or clinician. The housing 402 may be configured to be positioned by the user such that the external stimulator 400 is proximate to or against an exterior surface (e.g., skin) of the patient proximate to the magnetic driver 102. For example, the housing 402 may be shaped as eye glasses or an earpiece which may be worn by the patient. In another example, the housing 402 may be coupled to clothing and/or embedded within a piece of clothing such as a hat, a scarf, a belt, an arm band, a wrist band, a knee brace, a leg band, a compression sleeve, and/or the like.

Optionally, the housing 402 may include a user interface component 418, such as a button, a tactile switch, and/or the like on the surface of the housing 402, such as shown in FIG. 4. The user interface component 418 may be configured to activate and/or de-activate the external stimulator 400 device 102. For example, when the external stimulator 400 is positioned proximate to the lead 100 a user (e.g., clinician, patient) may turn on the external stimulator 400 via the user interface component 418 to generate the magnetic field from the ETC 412.

The ETC 412 may be an electrical conductor or a wire (e.g., thirty gauge to forty five gauge) composed of an electrically conductive material such as copper, gold, graphene, aluminum, nickel, and/or the like. The ETC 412 may have a diameter of about four to six millimeters and a length of about two to three centimeters. Optionally, the ETC 412 may have dimensions approximately the same and/or greater than the ERC 206. The ETC 412 may include multiple loops or turns (e.g., one hundred to two hundred) forming, for example, a coil or solenoid. Optionally, the ETC 412 may be helically wound about a rod (not shown). The rod may be configured to increase a magnitude of a magnetic field generated by the ETC 412. For example, the rod may provide a core for the ETC 412. The ETC 412 may be wound about the outer surface area of the rod composed of a ferrous material such as iron compounds or alloys, ferrites, and/or the like.

The ETC 412 may generate a magnetic field defined by one or more field characteristics in connection with a stimulation waveform. The field characteristics may correspond to a magnitude and/or direction of the magnetic field generated by the ETC 412 over time. The field characteristics are based on a current flowing through the ETC 412 based on an electrical potential and/or electrical signal from the generating circuitry 410.

The generating circuitry 410 may be configured to drive current with predetermined attributes to the ETC 412 resulting in a magnetic field having field characteristics that may provide power to the magnetic driver 102 to generate the stimulation waveform. The generating circuitry 410 may include one or more transistors, diodes, oscillators, amplifiers, and/or the like, which define the field characteristics of the magnetic field generated by the ETC 412. For example, the generating circuitry 410 may output an electrical potential across the ETC 412 resulting in a current therein. The current is based on the attributes (e.g., amplitude, frequency of pulses, pulse widths, number of pulses) of the electrical potential over time. The changes in current from the electrical potential define parameters of the magnetic field corresponding to the field characteristics. For example, a large current through the ETC 412 may correspond to a higher magnetic flux or magnitude of the magnetic field relative to a smaller current through the ETC 412.

The attributes of the stimulation waveform used by the generating circuitry 410 may be received and/or determined by the controller 408.

The controller 408 may include a microcontroller, a microprocessor, and/or one or more processors executing programmed instructions for controlling the various components of the external stimulator 400. Software or firmware code may be stored in the memory 414 of the external stimulator 400 or integrated with the controller 408. Additionally or alternatively, the controller 408 may include an ASIC, a programmable logic device, one or more differential amplifiers (e.g., comparators), and/or the like dedicated hardware components for performing one or more operations describe herein.

In various embodiments, the controller 408 may output attribute instructions to the generating circuitry 410 to create the magnetic field. For example, the controller 408 may access a desired stimulation waveform for the lead 100 to stimulate the target peripheral nerve. Based on the stimulation waveform, the controller 408 may determine field characteristics of the magnetic field, which will need to be created by the ETC 412 to provide the one or more pulses forming the stimulation waveform to the magnetic driver 102. The controller 408 may calculate attributes based on the determined field characteristics, and output the attributes to the generating circuitry 410. Additionally or alternatively, the attributes may be stored on the memory 414 and accessed by the controller 408.

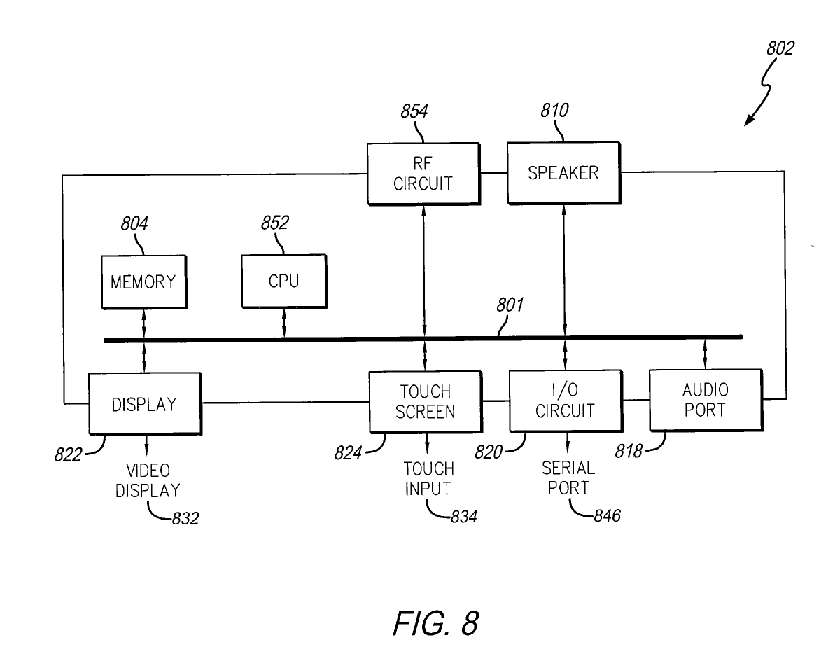

Optionally, the stimulation waveform may be selected from a stimulation waveform database stored in the memory 414. The stimulation waveform database may include a plurality of candidate stimulation waveforms stored in the memory 414. For example, the controller 408 may select a stimulation waveform from the stimulation waveform database based on an instruction signal received from a portable device 802 (FIG. 8) via the RF circuitry 406 and/or an I/O port 422.

The RF circuit 406 may include a transceiver or transmitter-receiver that includes an oscillator, a modulator, a demodulator, one or more amplifiers, an impedance circuit, and/or the like. The RF circuit 406 may allow the external stimulator 400 to establish a bi-directional communication link using a wireless protocol such as BLE, Bluetooth, ZigBee, and/or the like via the antenna 404 to receive the field characteristics and/or the stimulation waveform.

The antenna 404 may be an omnidirectional antenna such that the antenna 404 radiates and/or receives RF electromagnetic fields uniformly or equally in all directions. Thereby, the antenna 404 may transmit and/or receive wireless communications equally without limiting a position of the external stimulator 400. The antenna 404 may be tuned to a predetermined resonant frequency such that the antenna 404 has a signal performance exhibiting a lower return loss at a predetermined resonant frequency relative to alternative frequencies, such as a resonant frequency of the wireless protocol. For example, the wireless protocol may correspond to the Bluetooth low energy (BLE) protocol that operates within a 2.4 GHz band. The antenna 404 may be configured with the resonant frequency based on a shape of the antenna 404 (e.g., length, cross-sectional thickness, area) and/or by coupling components to the antenna 404 (e.g., capacitor, inductor) to achieve the resonant frequency of 2.4 GHz.

The I/O port 422 may be configured to receive an analogue and/or digital signal via a physical medium (e.g., cable, wire). For example, the I/O port 422 may include a physical connector configured to receive the physical medium, such as, an electric connector, a phone connector or “stereo jack” (e.g., TRS connector, TRRS connector, audio connector), a universal serial bus (USB) connector, and/or the like. Optionally, the I/O port 422 may correspond to defined communication protocol compatible with the controller 408. For example, the I/O port 422 may correspond to an I2C protocol, USB protocol, and/or the like. The I/O port 422 enables the external stimulator 400 to receive data along a physical medium and be physically coupled to a remote device (e.g., the portable device 802). For example, the external stimulator 400 may receive the instruction signal that provides the stimulation waveform or attributes of the stimulation waveform along a physical medium such as a cable via the I/O port 422 from the portable device.

Optionally, in various embodiments, one or more components of the external stimulator 400 may be integrated with the controller 408 to form a system on chip. (SoC). The SoC may be an integrated circuit (IC) such that all components of the SoC are on a single chip substrate (e.g., a single silicon die, a chip). For example, the SoC may have the memory 414, the controller 408, the RF circuit 406, and/or generating circuitry 410 embedded on a single die contained within a single chip package (e.g., QFN, TQFP, SOIC, BGA, and/or the like).

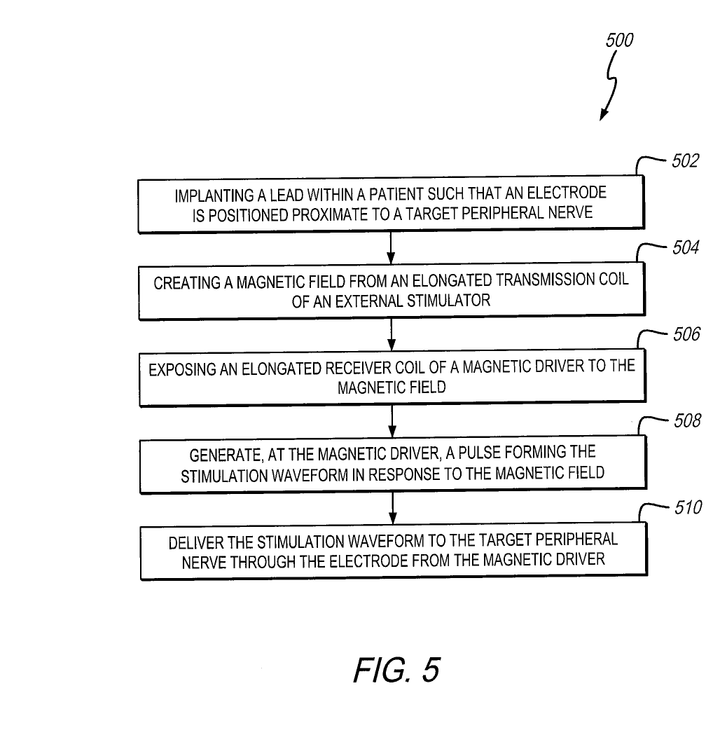

FIG. 5 is a flowchart of a method 500 for NS of peripheral nerve fibers. The method 500 may employ structures or aspects of various embodiments (e.g., systems and/or methods) discussed herein. Optionally, the operations of the method 500 may represent actions to be performed by one or more circuits (e.g., the magnetic driver 102, the controller 408) that include or are connected with processors, microprocessors, controllers, microcontrollers, Application Specific Integrated Circuits (ASICs), Field-Programmable Gate Arrays (FPGAs), or other logic-based devices that operate using instructions stored on a tangible and non-transitory computer readable medium (e.g., a computer hard drive, ROM, RAM, EEPROM, flash drive, or the like), such as software, and/or that operate based on instructions that are hardwired into the logic of the. For example, the operations of the method 500 may represent actions of or performed by one or more processors when executing programmed instructions stored on a tangible and non-transitory computer readable medium.

In various embodiments, certain steps (or operations) may be omitted or added, certain steps may be combined, certain steps may be performed simultaneously, certain steps may be performed concurrently, certain steps may be split into multiple steps, certain steps may be performed in a different order, or certain steps or series of steps may be re-performed in an iterative fashion. It should be noted, other methods may be used, in accordance with embodiments herein.

One or more methods may (i) create a magnetic field from an elongated transmission coil (ETC) of an external stimulator, (ii) expose an elongated receiver coil (ERC) of a magnetic driver to the magnetic field, (iii) generate, at the magnetic driver, a pulse forming a stimulation waveform in response to the magnetic field, and (iv) deliver the stimulation waveform to a target peripheral nerve through an electrode from the magnetic driver.

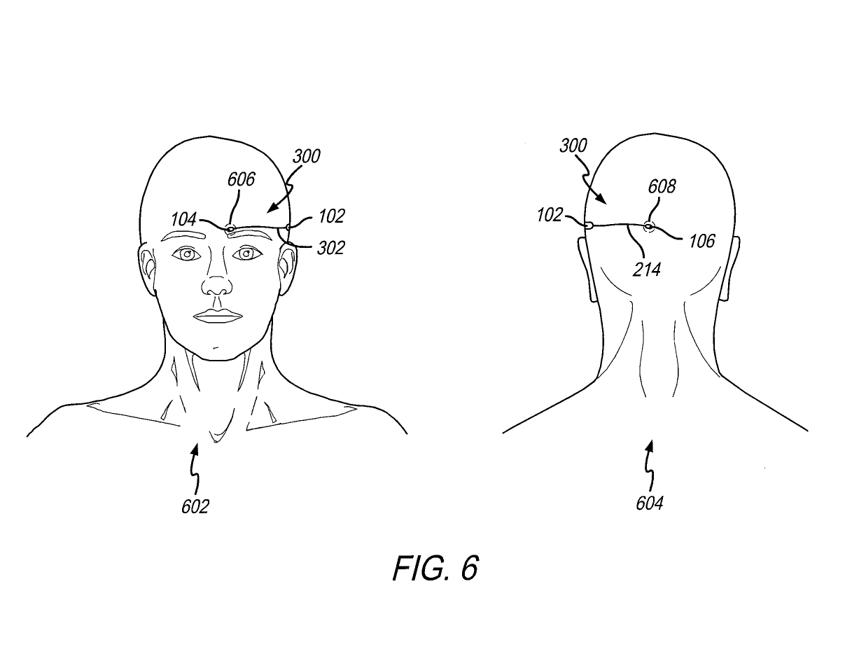

Beginning at 502, a lead (e.g., the lead 100, the lead 300) is implanted within a patient such that an electrode (e.g., 104, 106) is positioned proximate to a target peripheral nerve. The electrode is positioned with respect to the target peripheral nerve such that the one or more pulses emitted from the electrode stimulate the target peripheral nerve. For example, the electrode may be positioned within ten millimeters of the target peripheral nerve. It should be noted that in other embodiments, the electrode may be positioned closer than ten millimeters (e.g., five millimeters) or greater than ten millimeters (e.g., twenty millimeters, fifty millimeters). Additionally or alternatively, in connection with FIG. 6, the electrodes 104, 106 may be positioned proximate to two different target peripheral nerves.

FIG. 6 illustrates the lead 300 implanted within the patient. The lead 300 is shown in a patient anterior view 602 and a patient posterior view 604. The lead 300 is such that the electrodes 104 and 106 are positioned proximate to two target peripheral nerves, the supraorbital nerve 606 and the occipital nerve 608. For example, the electrode 104 is positioned proximate to the supraorbital nerve 606, and the electrode 106 is positioned proximate to the occipital nerve 608. The relative positions of the electrodes 104 and 106 allow the one or more pulses emitted by the electrodes 104 and 106 to activate and/or stimulate the target peripheral nerves 606 and 608, respectively.

The lead 300 may be implanted using a subcutaneous procedure. For example, a plurality of insulated needles may be positioned on the patient at various candidate locations of the target peripheral nerve. Insulated needles may be selected in an iterative process to verify that an insulated needle is located at the target peripheral nerve. The locations are verified based on patient feedback (e.g., verifying paresthesia) or based on autonomous reflexes by the patient during stimulation (e.g., blinking reflex activation). For example, a clinician may select one of the insulated needles. The selected insulated needle emits one or more pulses which form the stimulation waveform. If the patient senses paresthesia corresponding to stimulation of the target peripheral nerve, the location of the selected insulated needle is verified.

When a location is verified, the lead 300 may be implanted and positioned such that an electrode (e.g., the electrode 106, the electrode 104) is positioned proximate to the target peripheral nerve. For example, a suture (e.g., No. 2 suture, No. 3 suture) may be tunneled under the skin to a location behind the ear. The electrodes 104 and 106 are tied to the suture. The lead is pulled under the skin until the electrodes 104 and 106 reach the verified locations. Optionally, when the electrodes 104 and 106 are positioned at the verified locations, the magnetic driver 102 may deliver one or more pulses to the electrodes 104 and 106 to confirm the implantation location of the electrodes 104 and 106 stimulate the target peripheral nerves 606 and 608, respectively. The magnetic driver 102 may be implanted through an incision (e.g., three millimeter in length) and closed with a tissue adhesive.

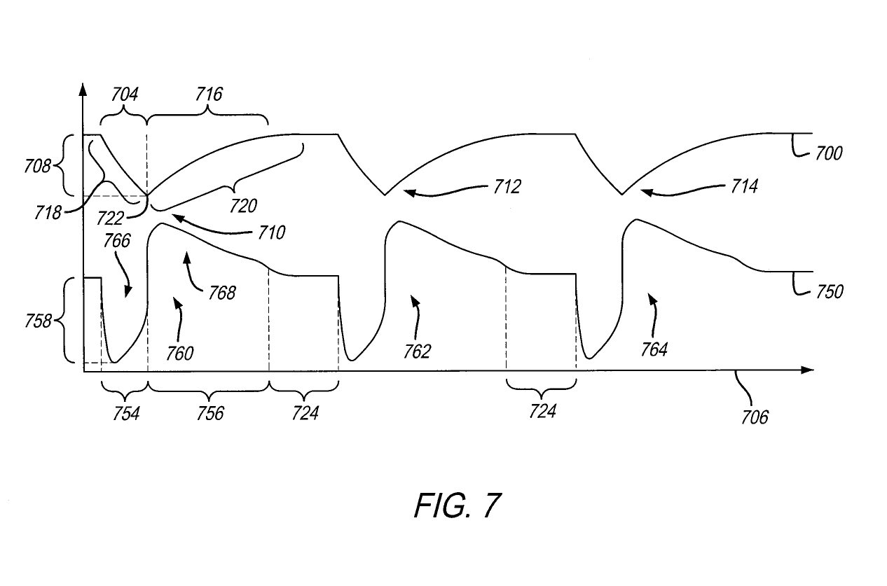

Returning to FIG. 5, at 504, a magnetic field is created from an ETC 412 of the external stimulator 400. For example, the external stimulator 400 may be positioned proximate to the lead 300. A user may activate the external stimulator 400 via the user interface component 418. Once activated, in connection with FIG. 7, the controller 408 may instruct the generating circuitry 410 to output and/or drive a current signal 700 to the ETC 412 over time.

FIG. 7 is a graphical representation of the current signal 700 received by the ETC 412 plotted over a horizontal axis 706 representing time. The current signal 700 is shown concurrently with a stimulation waveform 750 delivered by the magnetic driver 102 resulting from the current signal 700, as further described below.

The current signal 700 includes a series of pulses 710-714 each having an amplitude 708 separated by a pulse delay 724. The pulses 710-714, the morphology of the pulses 710-714, and the amplitude 708 result in field characteristic of the magnetic field in connection with the stimulation waveform 750 delivered by the magnetic coil 102 to the electrodes 104 and 106. For example, the number of pulses 710-714 of the current signal 700 corresponds to a number of pulses 760-764 of the stimulation waveform 750 delivered by the magnetic diver 102. It should be noted that in other embodiments the current signal 700 may have more than three pulses or less than three pulse (e.g., one pulse). In another example, a frequency of the pulses 710-714 corresponds to a frequency of the pulses 760-764.

An arrangement of the pulses 710-714 of the current signal 700 may form a stimulation pattern (e.g., tonic pattern, burst pattern, individual pulses, random/pseudorandom pulse trains) of the stimulation waveform 750. For example, to form a stimulation waveform 750 having a burst pattern, the pulses 710-714 may be grouped into a pulse train with a pulse delay 724 of one millisecond. The pulse train may be repeated by the external stimulator 400 every forty milliseconds. In another example, the pulse delay 724 may be adjusted by the controller 408 after each pulse 710-714 to form a random/pseudorandom pulse pattern of the stimulation waveform 750.

The morphology of the pulses 710-714 may correspond to characteristics of slopes 718, 720 forming the pulses 710-714 and/or a frequency of the pulses 710-714. For example, the duration 704 of the slope 718 from a baseline to a peak 722 of the pulse 710 corresponds to a pulse width 754 of a negative phase 766 of the pulse 760. In another example, the duration 716 of the slope 720 from the peak 722 to the baseline of the pulse 710 corresponds to a pulse width 756 of a positive phase of 768 of the pulse 760.

Generally, the current signal 700 controls field characteristics of the magnetic field in connection with the stimulation waveform 750. For example, as the current signal 700 passes through the ETC 412, a magnetic field is generated. The field characteristics of the magnetic field corresponds to a strength and/or direction of the magnetic field. The strength of the magnetic field may be associated with a magnitude of the current signal 700. For example, during the pulse 722, the strength of the magnetic field may be greatest at and/or near the peak 722 relative to other times during the pulse 722. The direction of the magnetic field may correspond to the slopes (e.g., 718, 720) of the current signal 700 associated with an electrical potential applied to the ETC 412. For example, the magnetic field generated by the ETC 412 during the slope 720 may have a direction different and/or opposite to the magnetic field generated by the ETC 412 during the slope 718.

Returning to FIG. 5, at 506, the ERC 206 of the magnetic driver 102 is exposed to the magnetic field. The ERC 206 may be exposed to the magnetic field generated by the ETC 412 when the ETC 412 or generally the external stimulator 400 is positioned proximate to the magnetic driver 102. For example, the external stimulator 400 may be positioned on an exterior surface of the patient (e.g., the skin) near the magnetic driver 102. Optionally, the ETC 412 may be placed within a few millimeters for the magnetic driver 102, for example, within ten millimeters.

At 508, a pulse (e.g., 760-762) forming the stimulation waveform 750 is generated at the magnetic driver 102 in response to the magnetic field. The stimulation waveform 750, shown in FIG. 7, may be generated by the ERC 206 as the ERC 206 is exposed and/or encounters the magnetic field outputted by the ETC 412. For example, the magnetic field induces a current through the ERC 206 resulting in a voltage signal corresponding to the stimulation waveform 750.

The pulses 760-762 of the stimulation waveform 750 are shown as biphasic pulses or charged balance, since the magnetic driver 102 does not carry a direct current. The phases of the pulses 760-762 are based on the field characteristics, such as the direction, of the magnetic field. For example, the slope 718 of the pulse 722 corresponds to the negative phase 766 having an amplitude 758. In another example, the slope 720 of the pulse 722 corresponds to the positive phase 768.

At 510, the stimulation waveform 750 is delivered to the target peripheral nerve through the electrode (e.g., 104, 106) from the magnetic driver 102. For example, the magnetic driver 102, specifically the ERC 206, is electrically coupled to the electrodes 104 and 106. As the stimulation waveform 750 is generated by the magnetic driver 102 in response to the magnetic field, the stimulation waveform 750 is conducted from the magnetic driver 102 to the electrodes 104 and 106, which emit the one or more pulses 760-764 forming the stimulation waveform 750.

Additionally or alternatively, the electrodes 104 and 106 may be electrically coupled to opposing terminals of the ERC 206 such that the magnitudes of the stimulation waveform 750 emitted by the electrodes 104 and 106 are reversed. For example, the electrodes 104 and 106 of the lead 300 may be electrically coupled to opposing terminals of the ERC 206. The stimulation waveform 750 is conducted from the magnetic driver 102 to the electrodes 104 and 106. The magnitudes of the stimulation waveform 750 emitted by the electrode 104 of the lead 300 may be similar to and/or the same as illustrated in FIG. 7. The magnitudes of the stimulation waveform 750 emitted by the electrode 106 of the lead 300 may be reversed such that a polarity of the negative phase 766 and positive phases 768 are switched.

As described above, in various embodiments, the external stimulator 400 may be provided the stimulation waveform within an instruction signal from the portable device 802.

FIG. 8 is a functional block diagram of the portable device 802, in accordance with an embodiment. The portable device 802 may be a smartphone, a tablet computer, a smartwatch, a laptop, and/or the like. A functional block diagram of the portable device 802, according to at least one embodiment, that is operated in accordance with the processes described herein and to interface with the external stimulator 400 as described herein.

The portable device 802 includes an internal bus 801 that may connect/interface with a Central Processing Unit (“CPU”) 852, memory 804, a speaker 810, a serial I/O circuit 820, a display 822, a touch screen 824, an audio port 818, and/or an RF circuit 854. The internal bus 801 may be an address/data bus that transfers information between the various components described herein. The memory 804 is a tangible and non-transitory computer readable medium, such as ROM, RAM, a hard drive, and/or the like. The memory 804 may store operational programs as well as data, such as current signal or stimulation waveform templates, algorithms for generating stimulation waveforms or current signals for the external stimulator 400, and/or the like. Additionally, the memory 804 may include programmed instructions representing actions for or performed by the CPU 852 when executing the programmed instructions.

Optionally, the serial bus 801 may connect/interface with other components, such as, a parallel I/O circuit, additional memory, additional user interface components (e.g., keyboard, tactile buttons, mouse), and/or the like.

The CPU 852 may typically include a microprocessor, a microcontroller, one or more processors, and/or equivalent control circuitry, designed specifically to control the portable device 802 and the external stimulator 400. The CPU 852 may include RAM, EEPROM, or ROM memory, logic and timing circuitry, state machine circuitry, and I/O circuitry to interface with the external stimulator 400.

The display 822 (e.g., may be connected to the video display 832) may be a liquid crystal display, a plasma display, and/or the like. The display 822 displays various information related to the processes described herein. The touch screen 824 may display graphic information relating to the external stimulator 400 (e.g., stimulation levels, stimulation waveforms) and include a graphical user interface (GUI). The GUI may include graphical icons, scroll bars, buttons, and the like which may receive or detect user or touch inputs 834 for the portable device 802 when selections are made by the user. Optionally the touch screen 824 may be integrated with the display 822. For example, the touch screen 824 may display the GUI allowing the user to enter and/or select current signals, stimulation waveforms, stimulation levels, and/or the like resulting in the instruction signal transmitted to the external stimulator 400.

The serial I/O circuit 820 interfaces with a serial port 846. The serial I/O circuit 820 may physically connect to the external stimulator 400 via the I/O port 422. Optionally, the serial I/O port may be coupled to a USB port or other interface capable of communicating with a USB device such as a memory stick.

Additionally or alternatively, the portable device 802 may wirelessly communicate with the external stimulator 400 a utilizing wireless protocol, such as Bluetooth, Bluetooth low energy, ZigBee, and/or the like. For example, the portable device 802 may communicate an instruction signal to the external stimulator. Optionally, the instruction signal may provide and/or include the stimulation waveform 750, the current signal 700, attributes of the stimulation waveform 750, and/or the like to the external stimulator 400 according to the wireless protocol. Additionally or alternatively, the instruction signal provide for a select stimulation waveform from a plurality of candidate stimulation waveforms stored on the memory 414 of the external stimulator 400.

Optionally, the instruction signal may include timing information and/or schedule of when the stimulation waveform 750 and/or select stimulation waveforms from the stimulation waveform database (e.g., stored on the memory 414) are to be emitted by the lead 100. For example, the instruction signal may designate a first stimulation signal to be emitted by the lead 100 at a first time period and second stimulation signal to be emitted by the lead at a second time period.

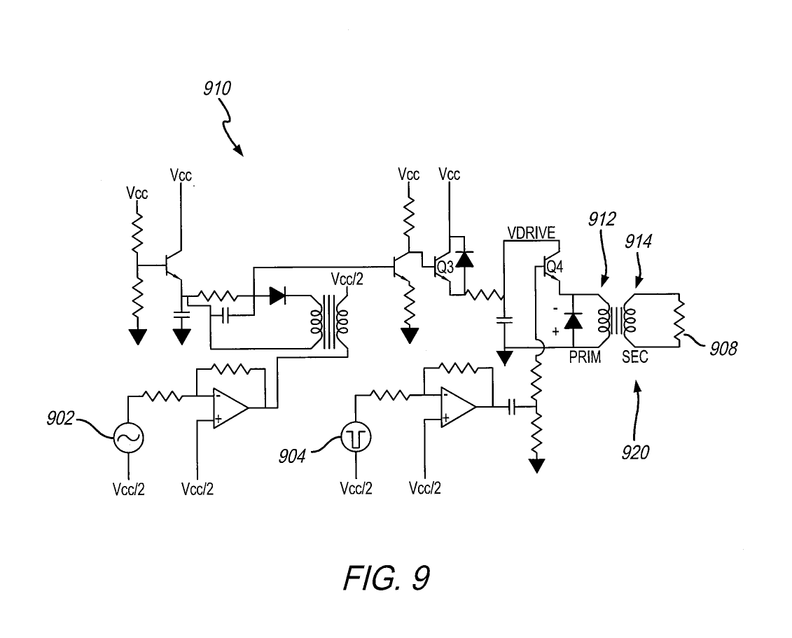

The audio port 818 may be an I/O interface for transmitting electrical signals along two stereo channels based on an audio file (e.g., MP3 file, Wave file). The audio port 818 may be an audio connector such as a stereo jack or “receive” phone connector. For example, a TRS connector, a TRRS connector, and/or the like. Optionally, in connection with FIG. 9, the portable device 802 may communicate with the external stimulator 910 (which may be similar to and/or the same as the external stimulator 400) via the audio port 818 through an audio connector. For example, the user may select an audio file via the GUI to play on the portable device 802. The audio file may correspond to attributes of the stimulation waveform 750, the stimulation waveform 750, attributes of the current signal 700, and/or the like, which are communicated to the external stimulator 910 and used to generate the magnetic field along a physical medium connected to the audio port 818.

FIG. 9 is a circuit diagram of an external stimulator 910 and a lead 920 in accordance with an embodiment. The lead 920, may be implanted subcutaneously within the patient, includes an ERC 914 that generates one or more pulses forming the stimulation waveform 750 in response to a magnetic field generated by an ETC 912 of the external stimulator 910. The ERC 914 may be similar to and/or the same as the ERC 206. The circuit diagram of the lead 920 includes a load 908 corresponding to tissue (e.g., the target peripheral nerve) of the patient.

The external stimulator 910 includes the ETC 912 that creates a magnetic field based on activation of a switch (e.g., transistor) Q4. The ETC 912 may be similar to and/or the same as the ETC 412. The external stimulator 910 may be connected to the portable device 400 along a physical medium such as a wire, cable, physical conductor, and/or the like. The physical medium may include an audio connector, such as a phone connector, which electrically and physically couples the external stimulator 910 to the portable device 802. For example, the physical medium include phone connectors positioned on opposing ends of the physical medium, and are connected and/or inserted into the audio port 818 of the portable device 802 and the I/O port (e.g., the I/O port 422) of the external stimulator 910.

The physical medium may include one or more electrically isolated channels, each carrying electrical signals that correspond to attributes of the stimulation waveform 750. The channels of the physical medium may correspond to the two stereo channels of the audio port 818 of the portable device 802 (FIG. 8). The physical medium carry information along the first and second channel corresponding to attributes of the stimulation waveform 750, and direct the external stimulator 910 on the field characteristics of the magnetic field generated by 912. For example, a reference waveform may be carried over the first channel and a control waveform over the second channel. The reference waveform may include modulation attributes, frequency attributes, and/or amplitude attributes of the stimulation waveform. The control waveform may include activation information corresponding to when the stimulation waveform occurs. Thereby, the reference waveform and the control waveform direct the external stimulator on attributes of the stimulation waveform.

In connection with FIG. 9, the external stimulator 910 receives two electrical signals 902 and 904 from the portable device 802 via the physical medium. The electrical signal 902 may be a sign wave having a frequency, for example, ranging from one to two kilohertz. Optionally, an amplitude of the sign wave may be adjusted based on an output volume of the portable device 802. The electrical signal 902 may correspond to the reference signal and is used to modulate the amplitude of the voltage supplied to the ETC 912 when the switch Q4 is activated. For example, the base voltage of Q3, which manages an amount of voltage supplied from the switch Q4 to the ETC 912, is controlled by the electrical signal 902.

The electrical signal 904, corresponding to a control signal, may be a negative pulse directing a duration of the pulses of the stimulation waveform 750 by activating/deactivating the switch Q4. For example, the negative pulse may have a pulse width of two hundred and fifty to five hundred microseconds. When the switch Q4 is activated by the negative pulse, the ETC 912 receives a voltage based on the electrical signal 902, and generates a magnetic field.

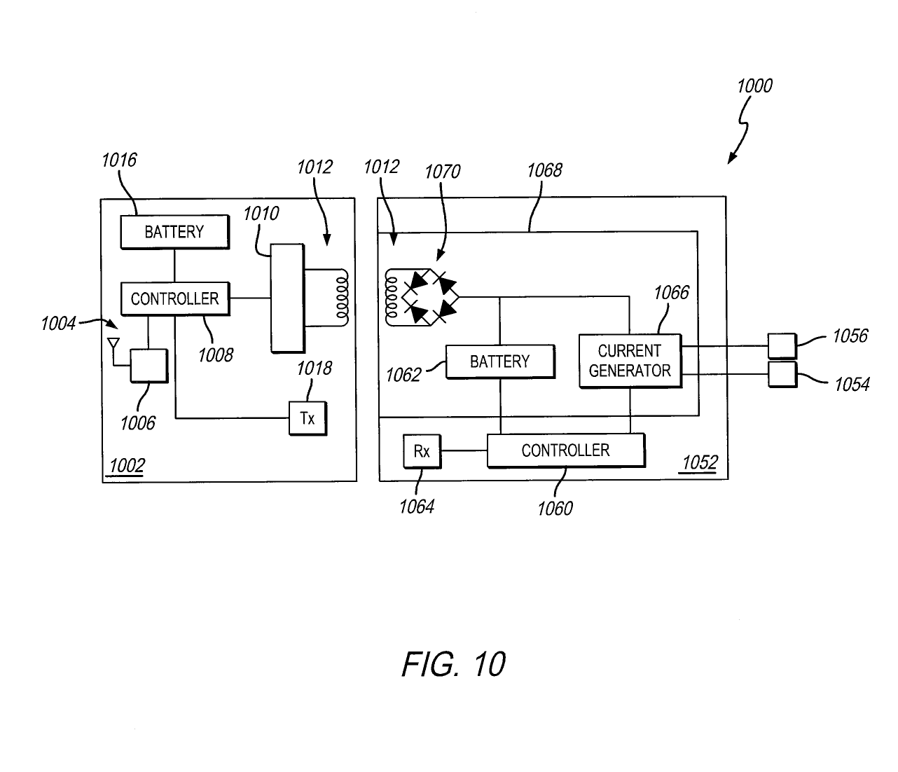

The leads 100, 300, and 920 are shown having an ERC 106, 914, respectively. Optionally, in connection with FIG. 10, additional components may be added to the lead using miniaturized electronics.

FIG. 10 is a schematic illustration of neurostimulation system 1000 that includes a lead 1052 and an external stimulator 1002. A magnetic driver 1068 of the lead 1052 may include an ETC 1058, a generator 1066, a battery 1062, and/or a bridge 1070 (e.g., bridge rectifier). The battery 1062 may have a small form factor and be rechargeable. For example, the battery 1016 may be a lithium battery having a charge of three to ten milliamp/hour.

The ETC 1058 is electrically coupled to the bridge 1070 which converts alternating current generated by the ETC 1058 in response to the magnetic field to a direct current, which can be used to charge the battery 1062, power a controller 1060, and/or drive the current generator 1066. The battery 1062 and the ETC 1058 may be electrically coupled to a current generator 1066 and a controller 1060. Optionally, the battery 1062 may provide supplement power when the ETC 1058 is not exposed to the magnetic field and/or does not supply enough power to the components of the lead 1052 (e.g., the controller 1060, the current driver 1066).

The current generator 1066 may include an amplifier, a transistor, a resistor, a capacitor, and/or the like configured to generate a stimulation waveform (e.g., the stimulation waveform 750 of FIG. 7). The current generator 1006 is electrically coupled to electrodes 1054 and 1056, which receive the stimulation waveform from the current generator 1066. The electrodes 1054 and 1056 may be similar to and/or the same as the electrodes 104 and 106 (FIGS. 1-3). The current generator 1066 may be controlled by a controller 1060.

The controller 1060 may include a microcontroller, a microprocessor, and/or one or more processors executing programmed instructions for controlling the constant generator 1066. For example, the controller 1060 may determine the frequency, amplitude, pulse width, stimulation pattern (e.g., tonic pattern, burst pattern) of the stimulation waveform, and/or the like outputted by the current generator 1066. Software or firmware code may be stored in memory (e.g., EEPROM) integrated with the controller 1060. Optionally, the controller 1060 may receive attributes of the stimulation waveform from the external stimulator 1002 via a receiver circuit 1064. The receiver circuit 1064 may include an antenna, one or more amplifiers, an impedance circuit, a communication coil for near-field or far-field communication, and/or the like.

Optionally, the lead 1052 may include a voltage multiplier. The voltage multiplier may include an amplifier, a capacitor, and/or diodes arranged to increase an output voltage of the battery 1062 and/or the bridge 1070 to the current generator 1066. For example, the voltage multiplier may raise the voltage above an output voltage of the battery 1062 by multiples of the battery voltage (e.g., two times, three times).

The external stimulator 1002 includes an ETC 1012, a power source (e.g., a battery) 1016, an RF circuit 1006, an antenna 1004, and generating circuitry 1010. The power source 1016 provides operating power to the controller 1008 and other components of the external stimulator 1002. The external stimulator 1002 may be similar to the external stimulator 400 (FIG. 4). For example, the generating circuitry 1010, the ETC 1012, the RF circuit 1006, and the antenna 1004 may be similar to and/or the same as the generating circuitry 410, the ETC 412, the RF circuit 406, and the antenna 405, respectively.

The controller 1008 may include a microcontroller, a microprocessor, and/or one or more processors executing programmed instructions for controlling the various components of the external stimulator 1002. Software or firmware code may be stored in memory 414 of the controller 408 (e.g., EEPROM). For example, the controller 1008 may execute programmed instructions that control the generating circuitry 1010 that provides voltage to the ETC 1012, which drives current through the ETC 1012 resulting in the magnetic field that provides power to the lead 1052.

The external stimulator 1002 may include a transmitter circuit 1018. The transmitter circuit 1018 may include may include an antenna, one or more amplifiers, an impedance circuit, a communication coil for near-field or far-field communication, and/or the like. For example, the controller 1008 may drive the communication coil of the transmitter circuit 1018 to transmit attributes of the stimulation waveform to the controller 1060 of the lead 1052.

Additionally or alternatively, the controller 1008 may communicate with the lead 1052 via the ETC 1002. For example, the controller 1008 may adjust a frequency of the voltage supplied to the ETC 1002, which adjusts field characteristics of the magnetic field (e.g., strength, direction). The changes to the frequency may be based on a frequency modulation communication scheme, such as a frequency-shift keying (FSK) modulation. For example, attributes of the stimulation waveform or the stimulation waveform is associated with the changes in frequency reflected in the field characteristics of the magnetic field.

The field characteristics may be measured by the controller 1060 of the lead 1052 by a sensing circuit (not shown) electrically coupled to the ERC 1058 and the controller 1060. For example, the sensing circuit may detect the frequency and/or changes in the frequency of current generated by the ERC 1058 in response to the magnetic field. The sensing circuitry may include op amps, transistors, logic gates, and/or the like.

It should be noted that the controllers 408, 1008, and 1060 and the CPU 852 may include any processor-based or microprocessor-based system including systems using microcontrollers, reduced instruction set computers (RISC), application specific integrated circuits (ASICs), field-programmable gate arrays (FPGAs), logic circuits, and any other circuit or processor capable of executing the functions described herein. Additionally or alternatively, the controllers 408, 1008, and 1060 and the CPU 852 may represent circuit modules that may be implemented as hardware with associated instructions (for example, software stored on a tangible and non-transitory computer readable storage medium, such as a computer hard drive, ROM, RAM, or the like) that perform the operations described herein. The above examples are exemplary only, and are thus not intended to limit in any way the definition and/or meaning of the term “controller.” The controllers 408, 1008, and 1060 and the CPU 852 may execute a set of instructions that are stored in one or more storage elements, in order to process data. The storage elements may also store data or other information as desired or needed. The storage element may be in the form of an information source or a physical memory element within the controllers 408, 1008, and 1060 and the CPU 852. The set of instructions may include various commands that instruct the controllers 408, 1008, and 1060 and the CPU 852 to perform specific operations such as the methods and processes of the various embodiments of the subject matter described herein. The set of instructions may be in the form of a software program. The software may be in various forms such as system software or application software. Further, the software may be in the form of a collection of separate programs or modules, a program module within a larger program or a portion of a program module. The software also may include modular programming in the form of object-oriented programming. The processing of input data by the processing machine may be in response to user commands, or in response to results of previous processing, or in response to a request made by another processing machine.

As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. The above memory types are exemplary only, and are thus not limiting as to the types of memory usable for storage of a computer program.

It is to be understood that the subject matter described herein is not limited in its application to the details of construction and the arrangement of components set forth in the description herein or illustrated in the drawings hereof. The subject matter described herein is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the invention without departing from its scope. While the dimensions, types of materials and coatings described herein are intended to define the parameters of the invention, they are by no means limiting and are exemplary embodiments. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. §112(f), unless and until such claim limitations expressly use the phrase “means for” followed by a statement of function void of further structure.

What is claimed is:

1. A neurostimulation system comprising:
an external stimulator having an elongated transmission coil configured to generate a magnetic field, wherein the external stimulator controls a field characteristic of the magnetic field in connection with a stimulation waveform; and
a lead implantable within a patient, the lead having a magnetic driver and an electrode, the magnetic driver being electrically coupled to the electrode, the magnetic driver including an elongated receiving coil that extends along an axis of the lead;
wherein, when the magnetic driver is exposed to the magnetic field, the magnetic driver generates a pulse forming the stimulation waveform to be delivered through the electrode to a target peripheral nerve.

2. The neurostimulation system of claim 1, wherein the target peripheral nerve corresponds to at least one of an occipital nerve, a supraorbital nerve, a trigeminal nerve, and a cervical nerve.

3. The neurostimulation system of claim 1, wherein the pulse is defined by at least one pulse characteristic that is based on the field characteristic of the magnetic field.

4. The neurostimulation system of claim 1, further comprising a portable device communicably coupled to the external stimulator, wherein the external stimulator receives an instruction signal from the portable device, the instruction signal providing the stimulation waveform.

5. The neurostimulation system of claim 4, wherein the portable device is a smartphone, a tablet, or a smartwatch.

6. The neurostimulation system of claim 4, wherein the instruction signal is communicated to the external stimulator along a physical medium.

7. The neurostimulation system of claim 6, wherein the physical medium has a first and second channel, a reference waveform is carried over the first channel and a control waveform over the second channel, the reference waveform and the control waveform direct the external stimulator on attributes of the stimulation waveform.

8. The neurostimulation system of claim 6, wherein the physical medium includes an audio connector such as a phone connector.

9. The neurostimulation system of claim 4, wherein the instruction signal is communicated to the external stimulator according to a wireless protocol, the wireless protocol constituting at least one of a Bluetooth protocol, a Bluetooth low energy protocol, and a Zigbee protocol.

10. The neurostimulation system of claim 1, wherein the magnetic driver includes a rod of a ferrous material, the elongated receiving coil is helically wound about the rod.

11. The neurostimulation system of claim 1, wherein the lead includes a receiver circuit communicably coupled to the external stimulator.

12. The neurostimulation system of claim 1, wherein the magnetic driver includes a current generator.

13. The neurostimulation system of claim 1, wherein the stimulation waveform is configured to relieve a migraine or headache.

14. The neurostimulation system of claim 1, wherein the external stimulator includes a housing, the housing coupled to an arm band, a leg band, a wrist band, a hat, a knee brace, a compression sleeve or an earpiece.

15. A method for neurostimulation of peripheral nerve fibers, the method comprising:
creating a magnetic field from an elongated transmission coil of an external stimulator, wherein the external stimulator controls a field characteristic of the magnetic field in connection with a stimulation waveform;
exposing an elongated receiver coil of a magnetic driver to the magnetic field;
generating, at the magnetic driver, a pulse forming the stimulation waveform in response to the magnetic field;
delivering the stimulation waveform to a target peripheral nerve through an electrode from the magnetic driver, wherein the magnetic driver is electrically coupled to the electrode.

16. The method of claim 15, further comprising transmitting characteristics of the stimulation waveform to the external stimulator from a portable device.

17. The method of claim 16, wherein the portable device is a smartphone, a tablet, or a smartwatch.

18. The method of claim 15, wherein the target peripheral nerve corresponds to at least one of an occipital nerve, a supraorbital nerve, a trigeminal nerve, and a cervical nerve.

19. The method of claim 15, wherein the pulse is defined by at least one pulse characteristic that is based on the field characteristic of the magnetic field.

20. The method of claim 15, further comprising implanting a lead within a patient such that the electrode is positioned proximate to the target peripheral nerve, wherein the lead includes the magnetic driver and the electrode.
